(19) United States
(12) Patent Application Publication
(10) Pub. No.: US 2024/0423149 A1
(43) Pub. Date: Dec. 26, 2024

(54) ENVIRONMENTAL-FRIENDLY AND EFFICIENT BREEDING METHOD OF HIGH-YIELD AND HIGH-QUALITY WHEAT CULTIVARS

(71) Applicant: Crop Research Institute, SAAS, Jinan (CN)

(72) Inventors: Jianmin SONG, Jinan (CN); Yan ZI, Jinan (CN); Jianjun LIU, Jinan (CN); Haosheng LI, Jinan (CN); Dungong CHENG, Jinan (CN); Aifeng LIU, Jinan (CN); Xinyou CAO, Jinan (CN); Jun GUO, Jinan (CN); Wei JU, Jinan (CN); Canguo WANG, Jinan (CN); Shengnan ZHAI, Jinan (CN); Faji LI, Jinan (CN); Cheng LIU, Jinan (CN); Ran HAN, Jinan (CN); Xiaolu WANG, Jinan (CN); Zhendong ZHAO, Jinan (CN)

(73) Assignee: Crop Research Institute, SAAS, Jinan (CN)

(21) Appl. No.: 18/482,845

(22) Filed: Oct. 6, 2023

(30) Foreign Application Priority Data
Jun. 25, 2023 (CN) 2023107554727

(51) Int. Cl. A01H 1/00; A01H 6/46

(52) U.S. Cl. CPC A01H 1/1245; A01H 1/121; A01H 1/1225; A01H 6/4678

(57) ABSTRACT

The present disclosure relates to the technical field of breeding, in particular to an environmental-friendly and efficient breeding method of high-yield and high-quality wheat cultivars. In the present disclosure, the breeding method can improve a breeding efficiency of excellent new cultivars, further realize large-scale production, and further increase a planting area of the excellent cultivars. In addition, the breeding method can increase the number of products for the new cultivars, further increase a diversity of early-generation materials, and further increase a diversity of hybrid samples through the innovation of hybridization and breeding methods. The environmental-friendly and efficient breeding method of high-yield and high-quality wheat cultivars can increase a richness of excellent characteristics of the new cultivars through high-generation materials, hybridization, and off-site identification, thereby further improving an effect of breeding.

### CROSS REFERENCE TO RELATED APPLICATION

This patent application claims the benefit and priority of Chinese Patent Application No. 2023107554727, filed on Jun. 25, 2023, the disclosure of which is incorporated by reference herein in its entirety as part of the present application.

### TECHNICAL FIELD

The present disclosure relates to the technical field of breeding, in particular to an environmental-friendly and efficient breeding method of high-yield and high-quality wheat cultivars.

### BACKGROUND

Breeding technology is to cultivating new varieties of excellent animals and plants by preparing genetic variation and improving genetic characteristics. The breeding technology is based on genetics, and comprehensively applies various disciplines such as ecology, physiology, biochemistry, pathology and biostatistics, which is of great significance to the development of animal husbandry and planting.

Wheat is an important staple crop in China. In recent years, with the change of climate and the decrease of arable land area, there is a greater downward pressure on staple food in China. Therefore, higher requirements are proposed for the breeding efficiency and effect. The existing wheat breeding technology mostly adopts a method of simple hybridization, and the selection of offspring is mostly based on the experience of breeders, without unified selection index and selection criterion. As a result, the existing technology has generally low breeding efficiency and poor effect, resulting in low output rate of new varieties; moreover, there are few excellent characteristics of the bred new varieties, which lag behind the needs of market diversification.

### SUMMARY

In order to solve the above problems, the present disclosure provides an environmental-friendly and efficient breeding method of high-yield and high-quality wheat cultivars. In the present disclosure, the breeding method provides different selection indexes and selection criteria according to different breeding objectives, showing a high breeding efficiency, and provides methods and approaches for breeding with multiple purposes.

To achieve the above objective, the present disclosure provides the following technical solutions:

The present disclosure provides an environmental-friendly and efficient breeding method of high-yield and high-quality wheat cultivars, including the following steps:
1) screening or preparing a breeding material according to a breeding objective; where traits of the breeding objective include one or more of super strong gluten, high-quality strong gluten, high-quality medium gluten, a high yield, a high water and fertilizer efficiency, disease resistance and stress resistance, a waxy property, and a high-resistant starch;
when the traits of the breeding objective include the super strong gluten or the high-quality strong gluten, traits of the breeding material include: a grain protein dry-basis content of greater than or equal to 14.0%, a wet gluten content of 14% wet-basis flour of greater than or equal to 31%, a gluten index of greater than or equal to 80%, a Zeleny sedimentation value of greater than or equal to 42 ml, a dough water absorption of greater than or equal to 62%, a stability time of greater than or equal to 12 min, a tensile area of greater than or equal to 100 cm², maximum resistance to extension of greater than or equal to 450 EU, an extensibility of greater than or equal to 160 mm, a loaf volume of greater than or equal to 850 mL, agronomic traits better than or equal to those of a high-yield control, and a yield greater than or equal to 10% of that of the high-yield control;
when the traits of the breeding objective include the high-quality medium gluten, the traits of the breeding material include: a grain protein dry-basis content of greater than or equal to 13.5%, a wet gluten content of 14% wet-basis flour of greater than or equal to 30%, a Zeleny sedimentation value of greater than or equal to 40 ml, a dough water absorption of greater than or equal to 60%, a stability time of greater than or equal to 5 min, a sensory score of noodles or steamed buns of greater than or equal to 85, agronomic traits equal to those of a high-yield control, and a yield greater than that of the high-yield control;
when the traits of the breeding objective include the high yield, the traits of the breeding material include: comprehensive traits better than those of a local dominant cultivar and a yield greater than or equal to 5% higher than that of the local dominant cultivar;
when the traits of the breeding objective include the disease resistance, the traits of the breeding material include: resistance to two or more major local diseases, better agronomic traits, and a yield greater than or equal to that of a high-yield control;
when the traits of the breeding objective include the high water and fertilizer efficiency and the stress resistance, the traits of the breeding materials include: comprehensive traits better than those of a local dominant cultivar, a yield greater than or equal to 5% higher than that of the local dominant cultivar, a water saving index of greater than or equal to 1.2, drought resistance of greater than or equal to 2, a nitrogen use efficiency of greater than or equal to 1.3, or a phosphorus use efficiency of greater than or equal to 1.1;
when the traits of the breeding objective include the waxy property, the traits of the breeding material include: an amylose content of less than 1%, an amylopectin content of greater than or equal to 99%, better agronomic traits, and a yield greater than or equal to 10% of that of a high-yield control; and
when the traits of the breeding objective include the high-resistant starch, the traits of the breeding material include: a resistant starch content of greater than 5.0%;
2) configuring a hybrid combination for the breeding material according to the breeding objective, and conducting hybridization to obtain an early-generation material; and
3) conducting selfing on the early-generation material for not less than five generations, and selecting high-yield and high-quality new wheat cultivars; where the new wheat cultivars and selection criteria thereof include one or more of the following items:
wheat with super strong gluten meeting the following criteria: a grain protein dry-basis content of greater than or equal to 14.5%, a wet gluten content of 14% wet-basis flour of greater than or equal to 33%, a gluten index of greater than or equal to 90%, a Zeleny sedimentation value of greater than or equal to 45 ml, a dough water absorption of greater than or equal to 63%, a stability time of greater than or equal to 15 min, a tensile area of greater than or equal to 110 cm², maximum resistance to extension of greater than or equal to 500 EU, an extensibility of greater than or equal to 170 mm, and a yield greater than or equal to 5% of that of a local dominant cultivar;
wheat with a high yield and high-quality strong gluten meeting the following criteria: a grain protein dry-basis content of greater than or equal to 14.0%, a wet gluten content of 14% wet-basis flour of greater than or equal to 31%, a gluten index of greater than or equal to 80%, a Zeleny sedimentation value of greater than or equal to 42 ml, a dough water absorption of greater than or equal to 62%, a stability time of greater than or equal to 12 min, a tensile area of greater than or equal to 100 cm², maximum resistance to extension of greater than or equal to 450 EU, an extensibility of greater than or equal to 160 mm, a loaf volume of greater than or equal to 800 mL, a loaf sensory score of greater than or equal to 80, and a yield greater than or equal to 5% of that of a local dominant cultivar;
wheat with a high yield and high-quality medium gluten meeting the following criteria: a grain protein dry-basis content of greater than or equal to 13.5%, a wet gluten content of 14% wet-basis flour of greater than or equal to 30%, a Zeleny sedimentation value of greater than or equal to 40 ml, a dough water absorption of greater than or equal to 60%, a stability time of greater than or equal to 5 min, a sensory score of noodles or steamed buns of greater than or equal to 85, and a yield of greater than or equal to that of a local dominant cultivar;
wheat with a high yield meeting the following criteria: a yield greater than or equal to 5% higher than that of a local dominant cultivar and a yield increase pilot rate of greater than or equal to 60%;
wheat with a high yield and disease resistance meeting the following criteria: resistance to two or more major local diseases and a yield greater than or equal to that of a local dominant cultivar;
wheat with a high water and fertilizer efficiency and stress resistance meeting the following criteria: a yield greater than or equal to 5% higher than that of a local dominant cultivar, a yield increase pilot rate of greater than or equal to 60%, a water saving index of greater than or equal to 1.2 for a water-saving cultivar, drought resistance of greater than or equal to level 2 for a drought-resistance cultivar, a nitrogen use efficiency of greater than or equal to 1.3 for a nitrogen-efficient cultivar, or a phosphorus use efficiency of greater than or equal to 1.1 for a phosphorus-efficient cultivar;
wheat with a waxy property meeting the following criteria: an amylose content of less than 2%, an amylopectin content of greater than or equal to 98%, and a yield greater than or equal to 5% higher than that of a local dominant cultivar; and
wheat with a high-resistant starch meeting the following criteria: a resistant starch content of greater than 4.0% and a yield greater than or equal to 5% higher than that of a local dominant cultivar.

Preferably, when the traits of the breeding objective include the high yield, in step 3), a process of the selecting includes: selecting a strain with a plant type of luxuriance and a grain-to-leaf ratio greater than or equal to 5% those of a local dominant cultivar.

Preferably, when the traits of the breeding objective include the high-quality strong gluten, in step 3), a process of the selecting includes:
measuring a sedimentation value of F1-generation seeds by a macro method, and selecting a strain with the sedimentation value of greater than or equal to 40 mL, a wet gluten content of 14% wet-basis flour of greater than or equal to 30%, and a protein content of greater than or equal to 13.5%;
measuring a sedimentation value of F2-generation single-plant seeds by a micro method, and selecting a strain with the sedimentation value of greater than or equal to 3.0 mL and a protein content of greater than or equal to 13.5%;
measuring a sedimentation value of F3-generation single-plant seeds by the micro method, and selecting a strain with the sedimentation value of greater than or equal to 3.2 mL and a protein content of greater than or equal to 14%; and
measuring a sedimentation value of F4-generation and subsequent-generations single-plant seeds by the micro method, and selecting a strain with the sedimentation value of greater than or equal to 3.4 mL and a protein content of greater than or equal to 14.5%.

Preferably, when the traits of the breeding objective include the high-quality medium gluten, in step 3), a process of the selecting includes:
selecting a strain with a gelatinization peak viscosity greater than or equal to 5% higher than that of a local high-quality dominant cultivar, which is not less than 2,900 cp; meanwhile, selecting a type with deletion of a Wx-B1 protein subunit, which has a total organic matter (TOM) value of 1.0 g±0.2 g.

Preferably, when the traits of the breeding objective include the disease resistance, in step 3), a process of the selecting includes:
selecting a strain with moderate resistance and higher resistance to two or more local diseases through composite identification.

Preferably, the composite identification includes one or more of natural diseases in the field, planting of infected lines or inoculation of infected strains in the field, and molecular marker-assisted selection.

Preferably, when the traits of the breeding objective include the high water and fertilizer efficiency and the stress resistance, in step 3), a process of the selecting includes:
setting a local dominant wheat cultivar with a high water and fertilizer efficiency and desirable stress resistance as a control cultivar, and selecting a cultivar with agronomic traits not less than those of the control cultivar and a yield and a water and fertilizer use efficiency greater than or equal to 5% better than those of the control cultivar.

Preferably, in step 3), the process of the selecting further includes: in comparison with the local dominant cultivar, selecting a strain with better spring puberty and more steady growth in an early stage, better fertility, a more stable yield, and better stress resistance in a middle stage, and a better leaf function and a higher stem quality in a later stage.

Preferably, in step 3), the process of the selecting further includes: conducting off-site identification and selection, and selecting a strain with traits better than those of dominant cultivars in different regions according to the breeding objective.

Preferably, in step 1), a process of screening or preparing a breeding material includes one or more of hybridization, selfing, marker-assisted selection, radiation mutagenesis, genetic engineering, chromosome engineering, and cell engineering.

### Beneficial Effects

The present disclosure provides an environmental-friendly and efficient breeding method of high-yield and high-quality wheat cultivars. In the present disclosure, the breeding method provides comprehensive application of multiple methods according to different breeding objectives, and provides methods and approaches for breeding with multiple purposes.

In the present disclosure, the breeding method shuttles among multiple breeding purposes and alternately selects multiple breeding methods, which is beneficial to breeding breakthrough cultivars with desirable comprehensive traits and multiple cultivars with an excellent individual trait.

In the present disclosure, the breeding method, through breeding material preparation and hybridization selection, can increase the number of products for the new cultivars, further increase a diversity of early-generation materials, and further increase a diversity of hybrid samples through the innovation of hybridization and breeding methods.

In addition, the breeding method can increase a richness of excellent characteristics of the new cultivars through high-generation materials, hybridization, and off-site identification, thereby further improving an effect of breeding.

### BRIEF DESCRIPTION OF THE DRAWINGS

To describe the technical solutions in the examples of the present disclosure or in the prior art more clearly, the accompanying drawings required for the examples will be briefly described below.

FIG. 1 shows a flowchart of a breeding method of the present disclosure.

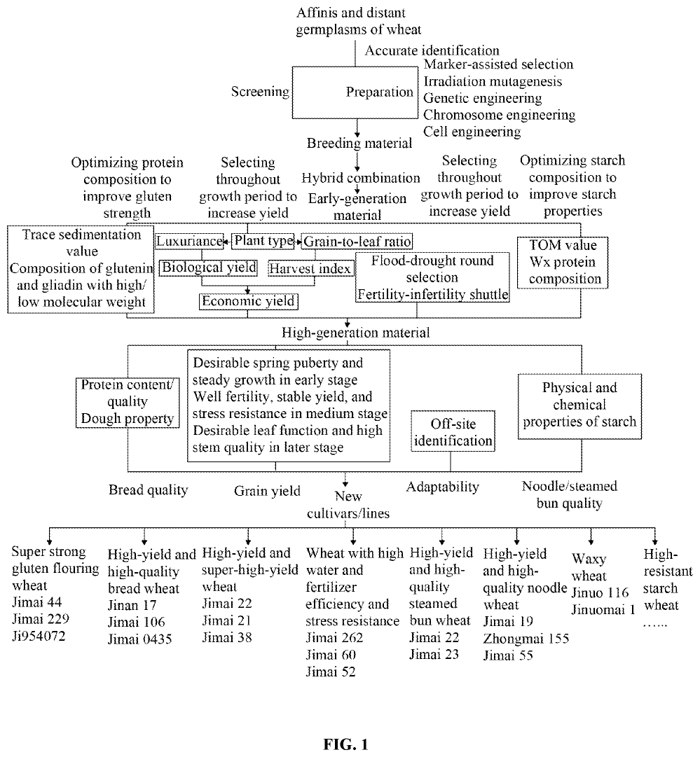

### DETAILED DESCRIPTION OF THE EMBODIMENTS

The present disclosure provides an environmental-friendly and efficient breeding method of high-yield and high-quality wheat cultivars, including the following steps:
1) screening or preparing a breeding material according to a breeding objective;
2) configuring a hybrid combination for the breeding material according to the breeding objective, and conducting hybridization to obtain an early-generation material; and
3) conducting selfing on the early-generation material for not less than five generations, and selecting high-yield and high-quality new wheat cultivars.

In the present disclosure, a breeding material is screened or prepared according to a breeding objective. A process of screening or preparing a breeding material includes preferably one or more of hybridization, selfing, marker-assisted selection, radiation mutagenesis, genetic engineering, chromosome engineering, and cell engineering.

In the present disclosure, traits of the breeding objective include one or more of super strong gluten, high-quality strong gluten, high-quality medium gluten, a high yield, disease resistance, a high water and fertilizer efficiency, stress resistance, a waxy property, and a high-resistant starch, more preferably a combination of the high yield with one or more of the above traits.

In the present disclosure, the control cultivar includes preferably a large-area local dominant cultivar; the high-yield control includes preferably a large-area and high-yield local dominant cultivar, or a control cultivar referenced to official regional trials, or a control cultivar set according to the breeding objective. The yield increase pilot rate is a percentage of pilot projects with increased yield in total pilot projects.
when the traits of the breeding objective include the super strong gluten or the high-quality strong gluten, traits of the breeding material include: a grain protein dry-basis content of greater than or equal to 14.0%, a wet gluten content of 14% wet-basis flour of greater than or equal to 31%, a gluten index of greater than or equal to 80%, a Zeleny sedimentation value of greater than or equal to 42 ml, a dough water absorption of greater than or equal to 62%, a stability time of greater than or equal to 12 min, a tensile area of greater than or equal to 100 cm², maximum resistance to extension of greater than or equal to 450 EU, an extensibility of greater than or equal to 160 mm, a loaf volume of greater than or equal to 850 mL, agronomic traits better than or equal to those of a high-yield control, and a yield greater than or equal to 10% of that of the high-yield control;
when the traits of the breeding objective include the high-quality medium gluten, the traits of the breeding material include: a grain protein dry-basis content of greater than or equal to 13.5%, a wet gluten content of 14% wet-basis flour of greater than or equal to 30%, a Zeleny sedimentation value of greater than or equal to 40 ml, a dough water absorption of greater than or equal to 60%, a stability time of greater than or equal to 5 min, a sensory score of noodles or steamed buns of greater than or equal to 85, agronomic traits equal to those of a high-yield control, and a yield greater than that of the high-yield control;
when the traits of the breeding objective include the high yield, the traits of the breeding material include: agronomic traits better than those of a local dominant cultivar and a yield greater than or equal to 5% higher than that of the local dominant cultivar;
when the traits of the breeding objective include the disease resistance, the traits of the breeding material include: resistance to two or more major local diseases, better agronomic traits, and a yield greater than or equal to that of a high-yield control;
when the traits of the breeding objective include the high water and fertilizer efficiency and the stress resistance, the traits of the breeding materials include: comprehensive traits better than those of a local dominant cultivar, a yield greater than or equal to 5% higher than that of the local dominant cultivar, a water saving index of greater than or equal to 1.2, drought resistance of greater than or equal to 2, a nitrogen use efficiency of greater than or equal to 1.3, or a phosphorus use efficiency of greater than or equal to 1.1;
when the traits of the breeding objective include the waxy property, the traits of the breeding material include: an amylose content of less than 1%, an amylopectin content of greater than or equal to 99%, better agronomic traits, and a yield greater than or equal to 10% of that of a high-yield control; and
when the traits of the breeding objective include the high-resistant starch, the traits of the breeding material include: a resistant starch content of greater than 5.0%.

In the present disclosure, a hybrid combination is configured for the breeding material according to the breeding objective, and hybridization is conducted to obtain an early-generation material.

In the present disclosure, selfing is conducted on the early-generation material for not less than five generations, and high-yield and high-quality new wheat cultivars are selected.

In the present disclosure, when the traits of the breeding objective include the high yield, a process of the selecting includes preferably: selecting a strain with a plant type of luxuriance and a grain-to-leaf ratio greater than or equal to 5% those of a local dominant cultivar; the process includes more preferably: selecting a high-yield cultivar or an ideal high-yield cultivar that has been popularized in a large area in production according to the breeding objective as a control cultivar, and selecting a strain with a plant type of luxuriance and a grain-to-leaf ratio greater than or equal to 5% those of the control cultivar; where the number of planting rows of the control cultivar and offsprings of the early-generation material is preferably 1:10; and Jimai 22 is selected as the control cultivar in the northern part of the Huanghuai winter wheat region and Shandong.

In the present disclosure, when the traits of the breeding objective include the high-quality strong gluten, a process of the selecting includes preferably:
measuring a sedimentation value of F1-generation seeds by a macro method, and selecting a strain with the sedimentation value of greater than or equal to 40 mL, a wet gluten content of 14% wet-basis flour of greater than or equal to 30%, and a protein content of greater than or equal to 13.5%;
measuring a sedimentation value of F2-generation single-plant seeds by a micro method, and selecting a strain with the sedimentation value of greater than or equal to 3.0 mL and a protein content of greater than or equal to 13.5%;
measuring a sedimentation value of F3-generation single-plant seeds by the micro method, and selecting a strain with the sedimentation value of greater than or equal to 3.2 mL and a protein content of greater than or equal to 14%; and
measuring a sedimentation value of F4-generation and subsequent-generations single-plant seeds by the micro method, and selecting a strain with the sedimentation value of greater than or equal to 3.4 mL and a protein content of greater than or equal to 14.5%; the process further includes preferably: selecting a single plant with 5+10 subunits as much as possible, and having as many high-quality and high-/low-molecular-weight glutenin and gliadin subunits as possible; where the single plant does not carry 1BL/1RS and other translocation fragments and genes that have a negative effect on quality. By increasing a selection standard generation by generation, more excellent strains can be retained for subsequent screening, thus avoiding premature elimination of the excellent strains.

In the present disclosure, when the traits of the breeding objective include the high-quality medium gluten, a process of the selecting includes preferably:
selecting a strain with a gelatinization peak viscosity greater than or equal to 5% higher than that of a local high-quality dominant cultivar, which is not less than 2,900 cp; meanwhile, selecting a type with deletion of a Wx-B1 protein subunit, which has a total organic matter (TOM) value of 1.0 g±0.2 g.

In the present disclosure, when the traits of the breeding objective include the disease resistance, a process of the selecting includes preferably:
selecting a strain with moderate resistance and higher resistance to two or more local diseases through composite identification; the composite identification includes one or more of natural diseases in the field, planting of infected lines or inoculation of infected strains in the field, and molecular marker-assisted selection; the process includes more preferably: determining a breeding objective of disease resistance according to local conditions; where for example, the Huanghuai winter wheat region requires that the five major diseases of powdery mildew, leaf rust, stripe rust, sheath blight, and wheat scab cannot all have high susceptibility, and the wheat is best to have moderate resistance and higher resistance to more than two diseases.

In the present disclosure, when the traits of the breeding objective include the high water and fertilizer efficiency and the stress resistance, a process of the selecting includes preferably:
setting a local dominant wheat cultivar with a high water and fertilizer efficiency and desirable stress resistance as a control cultivar, and selecting a cultivar with agronomic traits not less than those of the control cultivar and a yield and a water and fertilizer use efficiency greater than or equal to 5% better than those of the control cultivar; the process includes more preferably: when planting and selecting seeds in dry land, barren land, or water-saving conditions, setting corresponding control cultivars separately according to the breeding objective, such as planting a control cultivar with desirable drought resistance such as Lumai 21 when selecting seeds in the dry land, and planting a control cultivar with a high yield under water-saving conditions such as Jimai 22 when planting under the water-saving conditions; when selecting seeds on the barren land, planting a barren-resistant control cultivar; where selecting seeds, it is required that the agronomic traits are not less than those of the control cultivar and the yield and the water and fertilizer use efficiency are greater than or equal to 5% better than those of the control cultivar. Through flood-drought round selection and fertility-infertility shuttle of early-generation materials, high-generation wheat materials that have a high water and fertilizer efficiency and are water-saving and drought-resistant and adaptable to dry and barren land can be selected.

In the present disclosure, the process of the selecting further includes preferably: in comparison with the local dominant cultivar, selecting a strain with better spring puberty and more steady growth in an early stage, better fertility, a more stable yield, and better stress resistance in a middle stage, and a better leaf function and a higher stem quality in a later stage. When selecting, the strain and the control cultivar are strictly compared, and all indexes are required to be better than those of the control cultivar; 1 control cultivar can be selected, or different control cultivars can be selected according to different periods.

In the present disclosure, the process of the selecting further includes preferably: conducting off-site identification and selection, and selecting a strain with traits better than those of dominant cultivars in different regions according to the breeding objective; the process includes more preferably: arranging identification tests at representative locations in different ecological regions, requiring at least 70% of identification points, where the agronomic traits, yield, quality and other indexes of a selected material are better than those of the control cultivar.

In the present disclosure, the new wheat cultivars and selection criteria thereof include one or more of the following items:
wheat with super strong gluten meeting the following criteria: a grain protein dry-basis content of greater than or equal to 14.5%, a wet gluten content of 14% wet-basis flour of greater than or equal to 33%, a gluten index of greater than or equal to 90%, a Zeleny sedimentation value of greater than or equal to 45 ml, a dough water absorption of greater than or equal to 63%, a stability time of greater than or equal to 15 min, a tensile area of greater than or equal to 110 cm², maximum resistance to extension of greater than or equal to 500 EU, an extensibility of greater than or equal to 170 mm, and a yield greater than or equal to 5% of that of a local dominant cultivar;
wheat with a high yield and high-quality strong gluten meeting the following criteria: a grain protein dry-basis content of greater than or equal to 14.0%, a wet gluten content of 14% wet-basis flour of greater than or equal to 31%, a gluten index of greater than or equal to 80%, a Zeleny sedimentation value of greater than or equal to 42 ml, a dough water absorption of greater than or equal to 62%, a stability time of greater than or equal to 12 min, a tensile area of greater than or equal to 100 cm², maximum resistance to extension of greater than or equal to 450 EU, an extensibility of greater than or equal to 160 mm, a loaf volume of greater than or equal to 800 mL, a loaf sensory score of greater than or equal to 80, and a yield greater than or equal to 5% of that of a local dominant cultivar;
wheat with a high yield and high-quality medium gluten meeting the following criteria: a grain protein dry-basis content of greater than or equal to 13.5%, a wet gluten content of 14% wet-basis flour of greater than or equal to 30%, a Zeleny sedimentation value of greater than or equal to 40 ml, a dough water absorption of greater than or equal to 60%, a stability time of greater than or equal to 5 min, a sensory score of noodles or steamed buns of greater than or equal to 85, and a yield of greater than or equal to that of a local dominant cultivar;
wheat with a high yield meeting the following criteria: a yield greater than or equal to 5% higher than that of a local dominant cultivar and a yield increase pilot rate of greater than or equal to 60%;
wheat with a high yield and disease resistance meeting the following criteria: resistance to two or more major local diseases and a yield greater than or equal to that of a local dominant cultivar;
wheat with a high water and fertilizer efficiency and stress resistance meeting the following criteria: a yield greater than or equal to 5% higher than that of a local dominant cultivar, a yield increase pilot rate of greater than or equal to 60%, a water saving index of greater than or equal to 1.2 for a water-saving cultivar, drought resistance of greater than or equal to level 2 for a drought-resistance cultivar, a nitrogen use efficiency of greater than or equal to 1.3 for a nitrogen-efficient cultivar, or a phosphorus use efficiency of greater than or equal to 1.1 for a phosphorus-efficient cultivar;
wheat with a waxy property meeting the following criteria: an amylose content of less than 2%, an amylopectin content of greater than or equal to 98%, and a yield greater than or equal to 5% higher than that of a local dominant cultivar; and
wheat with a high-resistant starch meeting the following criteria: a resistant starch content of greater than 4.0% and a yield greater than or equal to 5% higher than that of a local dominant cultivar.

In the present disclosure, the breeding method can improve a breeding efficiency of excellent new cultivars, further realize large-scale production, and further increase a planting area of the excellent cultivars. In addition, the breeding method can increase the number of products for the new cultivars, further increase a diversity of early-generation materials, and further increase a diversity of hybrid samples through the innovation of hybridization and breeding methods. The breeding method for stress resistance of high-yield and high-quality wheat cultivars can increase a richness of excellent characteristics of the new cultivars through high-generation materials, hybridization, and off-site identification, thereby further improving an effect of breeding.

In order to further illustrate the present disclosure, the environmental-friendly and efficient breeding method of high-yield and high-quality wheat cultivars provided by the present disclosure are described in detail below in connection with examples, but these examples should not be understood as limiting the claimed scope of the present disclosure.

### Example 1

Breeding of a New Wheat Cultivar Jimai 22 with High Yield, Multiple Resistance, and Wide Applicability

### 1. Preparation of a Breeding Material (Parents)

The preparation of a male parent 935106 of the Jimai 22 had gone through three stages of gradient improvement:

(1) Preparation of 816134: a high-yielding, lodging-resistant, early-maturing, dry-heat-resistant strain of Taishan 5 was aggregated with a disease-resistant strain of Lovrin 13 to obtain an 816134 strain of wheat. This strain combined the high-yield characteristics of the female parent and the disease-resistant characteristics of the male parent, had a plant height of about 85 cm, desirable plant type, strong lodging resistance, well growth and appearance, disease resistance, heat resistance, excellent familiarity, large ears, and large grains; the disadvantages of this strain were common tillering ability, medium cold resistance, sparse spikelet arrangement, and slightly less grains per spike.

(2) Preparation of 865139: high-yield and stable-yield traits were further introduced, and multiple genes/traits of panicle and grain size of Bainong 77-4003 were aggregated to breed the 865139. This strain had luxuriant plants, desirable growth and appearance, with a plant height of about 90 cm, resistance to stripe rust and powdery mildew, and general tillering ability. However, this strain had a high rate of panicle formation, with about 320,000 panicles per mu, 20 spikelets, 3 infertile spikelets, about 32 grains per panicle, as well as large and plump grains, showing a thousand-grain weight of not less than 42 g. This strain had higher quality, a sedimentation value of about 30 ml (compared to 10 ml to 15 ml for Jinan 13), with a high and stable yield. In order to improve adaptability, 865139 had been identified in multiple places for two consecutive years. From 1988 to 1989, 5 test sites were selected: Jiaozhou City, Qingzhou City, Huantai County, Jining City, and Yanggu County; an average yield per mu of the genes in the 5 test sites was 345.6 kg, which was 7.7% higher than that of the control Jinan 13, ranking first among the 7 tested cultivars (lines). From 1989 to 1990, the appraisal continued at six sites in Linshu, Jiaozhou, Weibei, Jining, Huantai, and Yanggu; an average yield of 865139 was 355.6 kg/mu, with an average yield increase of 17.9% and a maximum yield increase of 46.8%, ranking 2rd among the 7 tested strains.

(3) Preparation of 935106: the high-yield and stable-yield traits were further improved, the 865139 was hybridized with 7588 ear/3-78-1-1-1, and then an obtained F1 was hybridized with Lumai 14 to introduce Lumai 14's wide adaptability and disease resistance traits, so as to obtain the 935106 strain of wheat. The 935106 strain had excellent plant type, vigorous growth, well appearance, short plant (72 cm), thick stalk, and high resistance to lodging. This strain showed desirable panicle traits, rectangular and large panicle shape, more than 32 grains per panicle, long awns, white shell, white grains, oval shape, large and plump grains, and a thousand-grain weight of 41 g. This strain had early maturation, and a heading stage 2 d earlier, a maturity period 1 day earlier, and a growth period 2 d shorter than those of Lumai 14; the strain also showed well comprehensive disease resistance, high resistance to powdery mildew, cold resistance, and excellent yellowing. This strain had excellent grain quality, a sedimentation value of about 36 ml, a wet gluten content of more than 37%, and excellent processing quality of steamed buns. This strain showed a high yield potential: from 1994 to 1995, it was identified that the strain had 361,000 spikes per mu, 32 grains per spike, a thousand-grain weight of 41 g, and a yield of 495.4 kg/mu, which was 8.9% higher than the adjacent control and 2.1% higher than the average control; from 1995 to 1996, it was identified that its F6-generation selection strain 946131 had 384,000 spikes per mu, a thousand-grain weight of 41.67 g, and a yield of 488.91 kg/mu; from 1996 to 1997, its F4-generation selection strain 924402 was identified and accepted in Longkou, with a yield of 642.18 kg per mu, becoming one of the few strains with a yield of more than 600 kg per mu in Shandong Province at that time.

The preparation of a female parent 935024 of the Jimai 22: a raw material was selected from a combination of Linyuan 7069/Lumai 14. This strain had well inherited the characteristics of the parent's multi-resistance, wide adaptability, desirable leaf function, and heat resistance in a later period. This strain showed a stronger tillering ability, a higher spike rate, with averagely 400,000 spikes per mu; this strain also had large and plump grains, spindle-shaped spikes, long awns, white shells, oval and white grains, cutin texture, and a thousand-grain weight of not less than 40 g. This strain had early maturity, with a heading stage 1 d earlier and a mature stage 2 d to 3 d earlier than those of the control Lumai 14; this strain also showed well comprehensive resistance, immunity to powdery mildew and stripe rust, desirable cold resistance, insensitivity to lack of light, strong growth in March, excellent yellowing, and wide adaptability. This strain had high yield and well yield stability: from 1993 to 1994, its F4-generation selection strain 924122 was identified to have the maximum yield among the 56 tested strains, with 400,000 spikes per mu, 29.5 grains per ear, a thousand-grain weight of 41.3 g, and a yield of 495.6 kg per mu, which was 18.9% higher than the adjacent control and 20.8% higher than the average control; from 1994 to 1995, it was identified that its F5-generation 935024 strain had 458,000 spikes per mu, which was the highest among the tested strains, and also had a thousand-grain weight of 43.9 g and a yield of 508.7 kg/mu, which were 9.9% and 4.8% higher than those of the adjacent control and the average control, respectively; from 1995 to 1996, it was identified that its F6-generation strain 946015 had a yield of 516.69 kg/mu, which was 7.64% higher than that of the adjacent control and 6.86% higher than that of the average control; from 1996 to 1997, a variety test showed that the average yield was 437 kg/mu, showing an increase of 7.1%.

### 2. Selection of Early-Generation Materials

The female parent of Jimai 22, 935024, had wide adaptability, multi-resistance, heat resistance, and high water and fertilizer efficiency. The male parent 935106 had desirable ear fertility and high yield potential, without heat resistance. The above two have outstanding advantages, rich genetic basis, few disadvantages, and strong complementary characters. In 1995, a hybrid combination was configured, numbered 95-86m.

In the selection of hybrid progeny: during the growth and development of wheat, the selection of luxuriance was emphasized in an early stage to increase a biological yield; the selection of stalk quality (elasticity and toughness) was emphasized in a medium stage to improve lodging resistance; and the selection of leaf function was emphasized in a later stage to improve heat resistance and ensure large and full grains.

From the F1-generation in 1995 to the F5-generation in 1999, this combination had outstanding performances, mainly including lush plants, well growth and appearance, short plants, desirable disease resistance, and full grains.

F1-generation in 1995: the combination showed well growth and appearance, full and large grains. The combination was selected for its overall desirable performance.

F2-generation in 1996: the combination was investigated as a key combination due to its overall desirable performance. In the field, the focus was on selection of plant height (dwarf stalk), panicle traits (large panicle and well fertility), and luxuriance; and strict selection was conducted on fullness of the grains after plant laboratory test, and 71 individual plants were selected.

F3-generation in 1997:71 selected individual plants were planted in different places in Heze (with frequent hot and dry wind) and Mengyin (with generally severe diseases), and then planted in dry land in Ningyang, followed by shuttle selection combined with flood-drought round selection. Due to the outstanding performance of this combination, in the F3-generation strain, 2 rows were planted for a single plant with better performance, and 4 rows were planted for a few key single plants, totaling 94 rows together with the remaining single plants. The individual plants in various places each performed well, with short stalks, disease resistance, compact plant type, and well leaf function, which were subjected to key selection. 120 individual plants were selected in the field, and 114 plants were selected after the plant laboratory test.

F4-generation in 1998:114 rows of this strain were planted, and 50 individual plants of 30 strains with desirable performance in various places were selected to continue the shuttle selection in different places and the flood-drought round selection. The results showed that the overall performance of the combination was outstanding, with short stalks, high elasticity, disease resistance, desirable plant type, small and uplifted leaves, and excellent leaf function in the later stage, but had dry tips and smaller spikes in some strains. The 34 strains with excellent performances were focused on the systemic production. The plant laboratory test results showed that the yield of these strains was significantly higher than that of the adjacent control (Lumai 14), and then 22 strains including the 984121 were remained for identification tests. While systemic production in the field was conducted, the excellent individual plants were also selected; based on the performance of various places, 20 strains were selected for further investigation and selection.

F5-generation in 1999: this combination still performed outstandingly, and 13 strains were produced by systemic production in the field (with a selection rate of 65%); after the plant laboratory test, 11 strains were selected for further investigation.

### 3. Identification of High-Generation Materials

In the identification test from 1999 to 2000, 22 strains of the 95-86m combination performed well overall, with strong tillering ability at the seedling stage, lush plants, high spike rate, disease resistance, dwarf stalks, desirable elasticity, lodging resistance, well appearance, excellent leaf function in the later period, desirable ripening, large and plump grain, and high yield; the yield of 18 strains increased compared with the average control, among which the yield of 4 strains ranked in the top 10 (there were a total of 327 identification materials, including the control). The yield per mu in the 984121 plot was 522.2 kg, showing an increase of 6.3% compared with the average control. After comprehensive consideration, it was recommended that 8 strains were promoted to the variety test.

In the identification test from 2000 to 2001, 11 strains of the F5-generation strain of the 95-86m combination still performed well, with lush plants, desirable plant type, and short plants, showing a plant height of 66 cm to 80 cm, averagely 71.9 cm, as well as a thousand-grain weight of 39.3 g, with 46 g as a maximum; these strains also had high yield potential, with an average yield of 536.87 kg/mu, which was 2.7% higher than that of the adjacent control and 6.73% higher than that of the average control; there were 5 sister strains with a yield of over 550 kg/mu, which was 9.3% to 19.3% higher than that of the average control. The 995001 had a yield ranking 1st among all the tested strains, with a plot yield of 600 kg/mu, and had a high quality, showing a water absorption of 59.4%, a formation time of 4 min, a stability time of 8 min, a softening degree of 40, and an evaluation value of 60.

In the variety test from 2000 to 2001, 8 strains of the 95-86m combination performed outstandingly overall, with averagely 414,900 spikes per mu, 32.41 grains per ear, and a thousand-grain weight of 40.81 g. The yield structure was coordinated, with an average yield of 463.34 kg/mu, which was 1.36% higher than that of the control. The 984121 strain had short, up-shooting leaves after rising, wider flag leaves after heading, darker appearance, larger spikelets, and a plant height of 70 cm, which grew neatly without powdery mildew and other diseases found in the field; this strain matured on June 2, with desirable ripening, full grains, and high quality; this strain also had a yield of 492.78 kg per mu, which was 7.51% higher than that of Lumai 14, ranking 5th among all tested strains. The 984119 strain had narrower leaves after rising, erect plants, wider flag leaves after heading, lighter color, and a plant height of 73 cm, without powdery mildew and other diseases found in the field; this strain matured on May 31, with desirable ripening; this strain also had a yield of 487.22 kg per mu, which was 6.30% higher than that of Lumai 14, ranking 7th among all tested strains.

In the variety test from 2001 to 2002, 5 strains of the F5-generation in the 95-86m combination performed well overall, with a plant height of 64 cm to 76 cm, averagely 70.8 cm, a maximum tiller of 1,325,400/mu, and an averagely 354,600 spikes per mu, reaching 385,000/mu as a maximum, averagely 28.8 grains per ear, reaching 36 grains as a maximum, an average thousand-grain weight of 44.4 g, reaching 50 g as a maximum. The number of grains per ear dropped more than that in last year, and the yield level was also declined, with an average yield of 460.94 kg/mu. The 995001 strain had semi-creeping seedlings, cold resistance, luxuriant plants, desirable plant type, relatively short plants, high lodging resistance, disease resistance, larger ears, horny and plump grains, and smooth skin; the yield in a plot was 513 kg/mu, which was 11.9% higher than that of Lumai 14, ranking the first.

In order to further investigate the adaptability of the 95-86m combination, 4 strains with high performance, such as 984121, were arranged for off-site identification in Heze, Mengyin, Lingxian, and Pingdu in 2000 to 2001, and then dry land identification in Ningyang. The results showed that 984121 performed outstandingly, with an average yield of 529.6 kg/mu, ranking first among the 20 tested strains; especially in Ningyang's dry land identification, the yield of this strain increased by 3.11%, showing a strong drought resistance potential.

### 4. Approval and Promotion

984121 participated in the regional test and production test in Shandong Province from 2003 to 2006; the strain passed the approval of Shandong Province in September 2006 (Lu Nongshen No. 2006050), named Jimai 22, and was planted in the middle and high fertilizer water plots of the whole province; the strain participated in the regional test and production test of the irrigated land group in the northern area of Huanghuai from 2004 to 2006, passed the national approval (National Approval Wheat 2006018) in January 2007 and was planted in Shandong, Hebei, Shanxi, and Henan; the strain was certified by Jiangsu Province in 2008 (Suyinmai 200801); the strain was certified by Anhui Province in March 2010 (Wan Nong, Nong Han No. 223); the strain was approved by Tianjin City in October 2010 (Jin Shenmai 2010005); the strain was granted the national plant variety right in May 2009 (variety right number CNA20060015.X).

Jimai 22 had high yield, stable yield, disease resistance and stress resistance, and wide adaptability. In the regional test in Shandong Province, the strain had an average yield of 537.04 kg/mu, which was 10.85% higher than that of the control; in 2009, experts organized by the Ministry of Agriculture in Tengzhou City, Shandong Province achieved an actual yield of 789.9 kg/mu, setting a record for the high yield of winter wheat under the double cropping system in China; the strain had prepared high-yield models in different ecological types for many years. According to the identification of the Chinese Academy of Agricultural Sciences, the strain had a new powdery mildew resistance gene (PmJM22), and was immune to powdery mildew, slowly responded to stripe rust, and was moderately resistant to moderately susceptible to stem rust, moderately susceptible to sheath blight, and then resistant to Sitodiplosis mosellana. The strain had strong drought resistance, with a drought resistance index of natural drought at 1.255 and a drought resistance index of simulated drought at 1.152, which was a cultivar having both high yield and high water use efficiency. The strain had lodging resistance, cold resistance, heat resistance, and wide adaptability, passed the examination (recognition) of the country and 6 provinces and cities including Shandong, and was promoted and planted across the southern, northern, and northern winter wheat areas of Huanghuai. The strain had a steamed bun sensory score of 87.1, with a fine texture, a desirable structure, a large volume, and an excellent processing quality.

### Example 2

### Breeding of High-Quality Noodle Wheat Jimai 19

### 1. Screening of a Breeding Material (Parents)

The agronomic traits (including grain-to-leaf ratio) and quality traits (determination of sedimentation value and wet gluten content for general resources, and farinograph test for special resources) were conducted on 260 germplasm resources; a TOM value of 102 materials was measured (with a variation range of 0.551 g to 3.773 g), and finally Lumai 13 and Linfen 5064 were selected for parent selection; the parents had large genetic differences and strong complementary traits.

The female parent Lumai 13 was a winter cultivar with strong cold resistance. Before the heading stage, the strain had stunted growth, excellent luxuriance, thick waxy stems and leaves, and dark green leaves; in the later stage, the strain had no resistance to dry and hot wind, poor yellowing, poor grain plumpness, a plant height of about 80 cm, resistance to fertilizer and lodging, high resistance to powdery mildew, resistance to stripe rust and leaf rust, and long awns. Lumai 13 had a sedimentation value of 35 ml to 40 ml, a wet gluten content of about 33.0%, a stability time of about 6.0 min, and a noodle TOM value of 1.435 g, which was an agronomic parent with desirable quality.

The male parent, Linfen 5064, was a semi-winter cultivar, with outstanding features of growth and development being rapid progress throughout the growth period (early, middle and late stages). The strain had well puberty growth and desirable field luxuriance at a jointing stage, but had poor luxuriance after heading due to the fast jointing-heading. The strain had early and fast grouting, strong resistance to cold damages in spring, dry-hot wind resistance, desirable yellowing, full grains, and high hardness. The strain showed a plant height of about 85 cm, well lodging resistance, moderate resistance to stripe rust, leaf rust and powdery mildew, and top awns. After years of testing, the Linfen 5064 had an average sedimentation value of 51 ml, a wet gluten content of 36.5%, a stability time of 11 min, and a noodle TOM value of 0.89 g. Due to the excellent heritability of high-quality genes, this strain was a rare high-quality parent material.

### 2. Selection of Early-Generation Materials

In 1989, a hybrid combination (Lumai 13/Linfen 5064) was configured, and the hybrid offspring were selected by a pedigree method.

From 1989 to 1990, the F1-generation strain showed a significant overall advantage, with luxuriant plants, well growth and appearance, relatively short plants, and desirable grain appearance quality.

From 1990 to 1991, the F2-generation strain had excellent overall performance, and had large variations in plant height, heading date, yellowing property, panicle number per plant, panicle traits, and powdery mildew resistance; therefore, this strain was listed as a key combination, and the agronomic traits and quality traits thereof were selected simultaneously. In the field, the selection of luxuriance, grain/leaf ratio, disease resistance, and yellowing was strengthened; plant laboratory test strictly selected the fullness of the grains, and then conducted micro-sedimentation tests. Finally, 15 plants were selected, and each plant had a sedimentation value of 2.8 ml to 5.2 ml (while the control Lumai 14 was 2.6 ml), and a grain-to-leaf ratio (a grain weight per spike/a sum of the area of the flag leaf and the second leaf) of averagely 61.6 mg/cm² (while the average of Lumai 14 was 56.2 mg/cm²), with an amplitude of variation of (54.6-72.4) mg/cm².

From 1991 to 1992, 15 strains of the F3-generation performed well in terms of growth, appearance, disease resistance, early maturity, and maturity; 8 strains were selected and retained in the field, and single-plant selection was conducted on the selected strains, and a total of 66 strains were selected; after plant laboratory test, grain identification, and quality analysis, 46 plants were finally selected, with a grain-to-leaf ratio of (62.4-68.2) mg/cm² (while the average of the control was 58.2 mg/cm²), and a micro-sedimentation value of 2.9 ml to 3.3 ml. The 9th strain among the 15 strains was more outstanding, with well growth and appearance, disease resistance, long functional period of leaves, good yellowing, high grain-to-leaf ratio (average 64.6 mg/cm²), and a heading stage 4 d earlier than that of the control (Lumai 14), such that 6 plants were selected for this strain.

From 1992 to 1993, the F4-generation continued to select individual plants. 45 strains were selected from 16 strains, with a grain-to-leaf ratio of (64.2-69.3) mg/cm² (while the average of the control was 55.8 mg/cm²), and a micro-sedimentation value of (3.09-3.35) ml.

From 1993 to 1994, the strain with line number 031 (935031) was systematically produced in the F5-generation. The results showed that this strain had a yield increase of 15.1% compared with the adjacent control, an average grain-to-leaf ratio of 68.9 mg/cm² (while the average of the control was 62.4 mg/cm²), a sedimentation value by the macro method of 40.2 ml, a sensory score in a noodle test of 93.2, and a TOM value of 1.08 g (while the control was 1.89 g). Therefore, the strain was recommended to participate in the next year's qualification test.

### 3. Identification of High-Generation Materials

In the identification test in 1994 to 1995, the yield per mu of plot 935031 was 569.7 kg, which was 23.1% and 17.4% higher than that of the adjacent control and the average control, ranking first among the tested strains; this strain had a sedimentation value of 42.0 ml, a wet gluten content of 35.7%, a stability time of 8.5 min, a softening degree of 56 BU, a noodle sensory score of 90.5 (compared to the sensory score of 78 for Lumai 14), and a TOM value of 1.12 g (compared to 1.79 g in the control). The strain had well plant luxuriance, high biological yield, coordinated three factors of the yield, and a grain-to-leaf ratio (an average of 20 panicles) of 46.4 mg/cm², compared to the adjacent control plot at 42.6 mg/cm²; the strain had 371,000 spikes per mu, 33.5 grains per ear, a thousand-grain weight of 44.5 g, a heading stage 3 d to 4 d earlier than that of the control, and a maturity period the same as that of the control.

From 1995 to 1996, 935031 participated in the variety test, with a grain-to-leaf ratio of 45.9 mg/cm², a yield of 516.5 kg/mu, with an increase of 10.9% compared with that of the control, a sedimentation value of 39.5 ml, a stability time of 7.0 min, a noodle sensory score of 89.5, and a TOM value of 1.02 g (compared to 1.89 g for the control).

### 4. Approval and Promotion

From 1996 to 2000, the 935031 participated in the (pre) test and production test in the high-fertilization area of Shandong Province. In May 2001, the strain was approved by the Shandong Provincial Crop Variety Approval Committee, named Jimai 19 (Lu Zhong Shen Zi [2001] No. 002), and promoted throughout the province.

From 1998 to 2001, the strain participated in the regional test and production test of the irrigated land group in the north of Huanghuai region. In 2003, the strain passed the national examination and approval (Guo Shen Mai 2003014).

Jimai 19 had an excellent plant type, high grain-to-leaf ratio, and high yield. In the high-fertilization regional test in Shandong Province from 1997 to 1999, the strain had an average two-year yield of 512.55 kg per mu, which was 5.65% higher than that of the control Lumai 14, ranking first; in the 1999-2000 production test, the yield of this strain was 508.6 kg per mu, which was 7.5% higher than that of the control Lumai 14.

Jimai 19 showed an excellent noodle processing quality. According to the quality analysis of the Grain Quality Supervision and Testing Center (Beijing) of the Ministry of Agriculture, this strain had a grain protein content (dry basis) of 14.65%, a wet gluten content of 35.2%, a sedimentation value of 38.6 ml, a water absorption of 68.2%, and a stability time of 6.9 min; the processed noodles were white and bright in color, smooth and delicate, moderate in hardness, toughness, and desirable in taste, with a sensory score of 93.5.

### Example 3

### Breeding of Super Strong Gluten Flouring Wheat, Jimai 229

### 1. Parent Selection

After years of identification, screening and preparation, it was decided to select high-quality strong gluten wheat Gaocheng 9411 as a female parent and 200040919 as a male parent, and a hybrid combination was configured in 2003.

Gaocheng 9411 (Gaocheng 8901/Annong 8455) had a semi-winter property, matured earlier, and showed a plant height of 70 cm, square ears, brown shells and short gray awns, strong gluten, and a stability time of farinograph of greater than 30 min; this strain also showed a sedimentation value of greater than 40 mL, carried high-quality glutenin subunits 5+10 with high molecular weight, had a thousand-grain weight of about 35 g, 49 grains per ear, and more than 7.5 million spikes per hectare, but had poor disease resistance and stress resistance.

200040919 was a high-quality and strong-gluten parent material prepared after years of improvement, with short stalks, large ears, lodging resistance, high-quality strong gluten, and a stability time of farinograph of not less than 20 min; the strain also had glutenin subunit compositions of 1, 7+9, and 5+10 with high molecular weight, but showed comprehensive agronomic traits to be further improved. Both parents showed excellent quality traits and strong complementary agronomic traits, making it easy to breed wheat cultivars with high-quality strong gluten, high yield, stable yield, disease resistance, and wide adaptability.

### 2. Selection of Early-Generation Materials

From 2004 to 2008, the selection was conducted according to a conventional pedigree method.

The F1 generation had neat field performance, desirable agronomic traits, outstanding high yield, and horny and plump kernels. 3280 plants were planted in the F2 generation, it was focused on selecting individual plants with well plant shape and desirable yellowing; the plant laboratory test was conducted to strictly screen the number of spikes and grain fullness, and a total of 67 individual plants were retained. From F3 to F5 generations, it was focused on selecting single plants in the field with well spring puberty, desirable plant type, strong disease resistance and stress resistance, excellent yellowing, large number of spikes, and high panicle fertility; the plant laboratory test was conducted to strictly select the single plants based on the number of grains per ear, grain size, color, plumpness, and uniformity as main indexes; meanwhile, combined with the results of protein content, sedimentation value, and kneading instrument indicators (with a grain protein content of greater than 16.0%, a sedimentation value of greater than 55.0 mL, and a kneading formation time of greater than 5.0 min), a single plant was selected. 50 strains were planted in the F6 generation, and lines with excellent, consistent, and stable agronomic traits were harvested and yielded. Two lines, 0860229 and 0860223, were selected through the results of sedimentation value measurement and kneading instrument analysis, and entered into the next year's identification test.

### 3. Selection of High-Generation Materials

In 2009, in the irrigated land identification test, the 0860229 strain had excellent comprehensive agronomic traits and quality traits, and entered the next year's variety test; in 2010, the strain participated in the variety test of Jinan and Mengyin varieties, and was excellent in both yield and quality.

### 4. Approval and Promotion

From 2011 to 2014, the 0860229 participated in the preliminary test, regional test, and production test of Shandong wheat high-fertilization group, and passed the approval of Shandong Province in 2015, which was named Jimai 229.

The quality analysis results of the wheat preliminary test and regional test in Shandong Province for three consecutive years showed that the Jimai 229 had excellent quality traits with little difference between years, showing an average bulk density of 806.67 g/L, a wet gluten content of 33.17%, a grain protein content of 15.53%, a sedimentation value of 45.10 mL, and a stability time of 23.67 min.

### Example 4

### Breeding of Super Strong Gluten Wheat, Jimai 954072

In the present disclosure, a famous high-quality wheat germplasm Lancota introduced from the United States was transformed; a high-generation material 80Q16-22-4-5 bred by the former Liaocheng Agricultural Sciences was used as a female parent, and Lancota was used as a male parent to prepare an intermediate material 884187; the material retained the quality characteristics of Lancota, and had significantly improved agronomic traits such as grain weight, yellowing property, and maturity. The 884187 had the nature of mid-winter, medium-early maturity, a plant height of 80 cm to 85 cm, no awn, resistance to dry and hot wind, high resistance to stripe rust, moderate resistance to powdery mildew, well yellowing property, and plump and hard grains. The strain had white grains, cutin texture, a thousand-grain weight of 45 g, a yield potential of 8350.5 kg·hm⁻², a grain protein content of 15.8%, a wet gluten content of 35.4%, a sedimentation value of 64 ml, a stability time of 25.7 min, and a starch gelatinization peak viscosity of 2,865 cP; the strain showed the HMW-GS compositions of 1, 13+16, and 5+10, the LMW-GS compositions of Glu-A3d and Glu-B3b, which was a non-1B/IR translocation line; this strain also had a loaf volume of 800 ml and a bread sensory score of 91, being rated as high-quality bread in the first national wheat quality appraisal. The wheat could not be used in large-scale production due to a highly compact plant type.

In addition, in the present disclosure, American high-quality strong gluten wheat Bag's for bread was transformed (as a basis/parent, the Bag's was hybridized with other materials, retaining its desirable traits, transforming its bad traits/introducing other desirable traits), so as to prepare an intermediate material 895392. The 895392 was hybridized with 884187, to breed 954072. This strain had a medium tillering ability, a plant height of 90 cm to 100 cm, high resistance to lodging, high resistance to stripe rust and leaf rust, moderate resistance to powdery mildew and head blight, and medium early maturity; the strain also showed a thousand-grain weight of 50 g, a yield potential of over 9,000 kg·hm⁻², the HMW-GS compositions of 1, 7+9, and 5+10, a protein content of 22.15%, and a loaf volume of 1,050 cm³. Therefore, this strain was an extremely rare high-protein, large-grain, and high-quality wheat with super strong gluten, and once became the first choice of wheat for processing enterprises and grain enterprises. A series of new wheat varieties such as Jimai 229, Jimai 44, and Shannong 20 were successively derived from the 954072, which provided abundant high-quality resources for the genetic improvement of wheat in China.

In summary, the breeding method can improve a breeding efficiency of excellent new cultivars, further realize large-scale production, and further increase a planting area of the excellent cultivars. In addition, the breeding method can increase the number of products for the new cultivars, further increase a diversity of early-generation materials, and further increase a diversity of hybrid samples through the innovation of hybridization and breeding methods. The breeding method for stress resistance of high-yield and high-quality wheat cultivars can increase a richness of excellent characteristics of the new cultivars through high-generation materials, hybridization, and off-site identification, thereby further improving an effect of breeding.

The present disclosure has been disclosed with preferred examples as above, which shall not be construed as a limitation to the present disclosure. Any person skilled in the art can make changes and variations without departing from the spirit and scope of the present disclosure. The present disclosure shall fall within the protection scope defined in the claims.

What is claimed is:

1. An environmental-friendly and efficient breeding method of high-yield and high-quality wheat cultivars, comprising the following steps:
1) screening or preparing a breeding material according to a breeding objective; wherein traits of the breeding objective comprise one or more of super strong gluten, high-quality strong gluten, high-quality medium gluten, a high yield, a high water and fertilizer efficiency, disease resistance and stress resistance, a waxy property, and a high-resistant starch;
when the traits of the breeding objective comprise the super strong gluten or the high-quality strong gluten, traits of the breeding material comprise: a grain protein dry-basis content of greater than or equal to 14.0%, a wet gluten content of 14% wet-basis flour of greater than or equal to 31%, a gluten index of greater than or equal to 80%, a Zeleny sedimentation value of greater than or equal to 42 ml, a dough water absorption of greater than or equal to 62%, a stability time of greater than or equal to 12 min, a tensile area of greater than or equal to 100 cm², maximum resistance to extension of greater than or equal to 450 EU, an extensibility of greater than or equal to 160 mm, a loaf volume of greater than or equal to 850 mL, agronomic traits better than or equal to those of a high-yield control, and a yield greater than or equal to 10% of that of the high-yield control;
when the traits of the breeding objective comprise the high-quality medium gluten, the traits of the breeding material comprise: a grain protein dry-basis content of greater than or equal to 13.5%, a wet gluten content of 14% wet-basis flour of greater than or equal to 30%, a Zeleny sedimentation value of greater than or equal to 40 ml, a dough water absorption of greater than or equal to 60%, a stability time of greater than or equal to 5 min, a sensory score of noodles or steamed buns of greater than or equal to 85, agronomic traits equal to those of a high-yield control, and a yield greater than that of the high-yield control;
when the traits of the breeding objective comprise the high yield, the traits of the breeding material comprise: comprehensive traits better than those of a local dominant cultivar and a yield greater than or equal to 5% higher than that of the local dominant cultivar;
when the traits of the breeding objective comprise the disease resistance, the traits of the breeding material comprise: resistance to two or more major local diseases, better agronomic traits, and a yield greater than or equal to that of a high-yield control;
when the traits of the breeding objective comprise the high water and fertilizer efficiency and the stress resistance, the traits of the breeding materials comprise: comprehensive traits better than those of a local dominant cultivar, a yield greater than or equal to 5% higher than that of the local dominant cultivar, a water saving index of greater than or equal to 1.2, drought resistance of greater than or equal to 2, a nitrogen use efficiency of greater than or equal to 1.3, or a phosphorus use efficiency of greater than or equal to 1.1;
when the traits of the breeding objective comprise the waxy property, the traits of the breeding material comprise: an amylose content of less than 1%, an amylopectin content of greater than or equal to 99%, better agronomic traits, and a yield greater than or equal to 10% of that of a high-yield control; and
when the traits of the breeding objective comprise the high-resistant starch, the traits of the breeding material comprise: a resistant starch content of greater than 5.0%;
2) configuring a hybrid combination for the breeding material according to the breeding objective, and conducting hybridization to obtain an early-generation material; and
3) conducting selfing on the early-generation material for not less than five generations, and selecting high-yield and high-quality new wheat cultivars; wherein the new wheat cultivars and selection criteria thereof comprise one or more of the following items:
wheat with super strong gluten meeting the following criteria: a grain protein dry-basis content of greater than or equal to 14.5%, a wet gluten content of 14% wet-basis flour of greater than or equal to 33%, a gluten index of greater than or equal to 90%, a Zeleny sedimentation value of greater than or equal to 45 ml, a dough water absorption of greater than or equal to 63%, a stability time of greater than or equal to 15 min, a tensile area of greater than or equal to 110 cm², maximum resistance to extension of greater than or equal to 500 EU, an extensibility of greater than or equal to 170 mm, and a yield greater than or equal to 5% of that of a local dominant cultivar;
wheat with a high yield and high-quality strong gluten meeting the following criteria: a grain protein dry-basis content of greater than or equal to 14.0%, a wet gluten content of 14% wet-basis flour of greater than or equal to 31%, a gluten index of greater than or equal to 80%, a Zeleny sedimentation value of greater than or equal to 42 ml, a dough water absorption of greater than or equal to 62%, a stability time of greater than or equal to 12 min, a tensile area of greater than or equal to 100 cm², maximum resistance to extension of greater than or equal to 450 EU, an extensibility of greater than or equal to 160 mm, a loaf volume of greater than or equal to 800 mL, a loaf sensory score of greater than or equal to 80, and a yield greater than or equal to 5% of that of a local dominant cultivar;
wheat with a high yield and high-quality medium gluten meeting the following criteria: a grain protein dry-basis content of greater than or equal to 13.5%, a wet gluten content of 14% wet-basis flour of greater than or equal to 30%, a Zeleny sedimentation value of greater than or equal to 40 ml, a dough water absorption of greater than or equal to 60%, a stability time of greater than or equal to 5 min, a sensory score of noodles or steamed buns of greater than or equal to 85, and a yield of greater than or equal to that of a local dominant cultivar;
wheat with a high yield meeting the following criteria: a yield greater than or equal to 5% higher than that of a local dominant cultivar and a yield increase pilot rate of greater than or equal to 60%;
wheat with a high yield and disease resistance meeting the following criteria: resistance to two or more major local diseases and a yield greater than or equal to that of a local dominant cultivar;
wheat with a high water and fertilizer efficiency and stress resistance meeting the following criteria: a yield greater than or equal to 5% higher than that of a local dominant cultivar, a yield increase pilot rate of greater than or equal to 60%, a water saving index of greater than or equal to 1.2 for a water-saving cultivar, drought resistance of greater than or equal to level 2 for a drought-resistance cultivar, a nitrogen use efficiency of greater than or equal to 1.3 for a nitrogen-efficient cultivar, or a phosphorus use efficiency of greater than or equal to 1.1 for a phosphorus-efficient cultivar;
wheat with a waxy property meeting the following criteria: an amylose content of less than 2%, an amylopectin content of greater than or equal to 98%, and a yield greater than or equal to 5% higher than that of a local dominant cultivar; and
wheat with a high-resistant starch meeting the following criteria: a resistant starch content of greater than 4.0% and a yield greater than or equal to 5% higher than that of a local dominant cultivar.

2. The breeding method according to claim 1, wherein when the traits of the breeding objective comprise the high yield, in step 3), a process of the selecting comprises: selecting a strain with a plant type of luxuriance and a grain-to-leaf ratio greater than or equal to 5% those of a local dominant cultivar.

3. The breeding method according to claim 1, wherein when the traits of the breeding objective comprise the high-quality strong gluten, in step 3), a process of the selecting comprises:
measuring a sedimentation value of F1-generation seeds by a macro method, and selecting a strain with the sedimentation value of greater than or equal to 40 mL, a wet gluten content of 14% wet-basis flour of greater than or equal to 30%, and a protein content of greater than or equal to 13.5%;
measuring a sedimentation value of F2-generation single-plant seeds by a micro method, and selecting a strain with the sedimentation value of greater than or equal to 3.0 mL and a protein content of greater than or equal to 13.5%;
measuring a sedimentation value of F3-generation single-plant seeds by the micro method, and selecting a strain with the sedimentation value of greater than or equal to 3.2 mL and a protein content of greater than or equal to 14%; and
measuring a sedimentation value of F4-generation and subsequent-generations single-plant seeds by the micro method, and selecting a strain with the sedimentation value of greater than or equal to 3.4 mL and a protein content of greater than or equal to 14.5%.

4. The breeding method according to claim 1, wherein when the traits of the breeding objective comprise the high-quality medium gluten, in step 3), a process of the selecting comprises:
selecting a strain with a gelatinization peak viscosity greater than or equal to 5% higher than that of a local high-quality dominant cultivar, which is not less than 2,900 cp; meanwhile, selecting a type with deletion of a Wx-B1 protein subunit, which has a total organic matter (TOM) value of 1.0 g±0.2 g.

5. The breeding method according to claim 1, wherein when the traits of the breeding objective comprise the disease resistance, in step 3), a process of the selecting comprises:
selecting a strain with moderate resistance and higher resistance to two or more local diseases through composite identification.

6. The breeding method according to claim 5, wherein the composite identification comprises one or more of natural diseases in the field, planting of infected lines or inoculation of infected strains in the field, and molecular marker-assisted selection.

7. The breeding method according to claim 1, wherein when the traits of the breeding objective comprise the high water and fertilizer efficiency and the stress resistance, in step 3), a process of the selecting comprises:
setting a local dominant wheat cultivar with a high water and fertilizer efficiency and desirable stress resistance as a control cultivar, and selecting a cultivar with agronomic traits not less than those of the control cultivar and a yield and a water and fertilizer use efficiency greater than or equal to 5% better than those of the control cultivar.

8. The breeding method according to claim 1, wherein in step 3), the process of the selecting further comprises: in comparison with the local dominant cultivar, selecting a strain with better spring puberty and more steady growth in an early stage, better fertility, a more stable yield, and better stress resistance in a middle stage, and a better leaf function and a higher stem quality in a later stage.

9. The breeding method according to claim 2, wherein in step 3), the process of the selecting further comprises: in comparison with the local dominant cultivar, selecting a strain with better spring puberty and more steady growth in an early stage, better fertility, a more stable yield, and better stress resistance in a middle stage, and a better leaf function and a higher stem quality in a later stage.

10. The breeding method according to claim 3, wherein in step 3), the process of the selecting further comprises: in comparison with the local dominant cultivar, selecting a strain with better spring puberty and more steady growth in an early stage, better fertility, a more stable yield, and better stress resistance in a middle stage, and a better leaf function and a higher stem quality in a later stage.

11. The breeding method according to claim 4, wherein in step 3), the process of the selecting further comprises: in comparison with the local dominant cultivar, selecting a strain with better spring puberty and more steady growth in an early stage, better fertility, a more stable yield, and better stress resistance in a middle stage, and a better leaf function and a higher stem quality in a later stage.

12. The breeding method according to claim 5, wherein in step 3), the process of the selecting further comprises: in comparison with the local dominant cultivar, selecting a strain with better spring puberty and more steady growth in an early stage, better fertility, a more stable yield, and better stress resistance in a middle stage, and a better leaf function and a higher stem quality in a later stage.

13. The breeding method according to claim 6, wherein in step 3), the process of the selecting further comprises: in comparison with the local dominant cultivar, selecting a strain with better spring puberty and more steady growth in an early stage, better fertility, a more stable yield, and better stress resistance in a middle stage, and a better leaf function and a higher stem quality in a later stage.

14. The breeding method according to claim 7, wherein in step 3), the process of the selecting further comprises: in comparison with the local dominant cultivar, selecting a strain with better spring puberty and more steady growth in an early stage, better fertility, a more stable yield, and better stress resistance in a middle stage, and a better leaf function and a higher stem quality in a later stage.

15. The breeding method according to claim 1, wherein in step 3), the process of the selecting further comprises: conducting off-site identification and selection, and selecting a strain with traits better than those of dominant cultivars in different regions according to the breeding objective.

16. The breeding method according to claim 2, wherein in step 3), the process of the selecting further comprises: conducting off-site identification and selection, and selecting a strain with traits better than those of dominant cultivars in different regions according to the breeding objective.

17. The breeding method according to claim 3, wherein in step 3), the process of the selecting further comprises: conducting off-site identification and selection, and selecting a strain with traits better than those of dominant cultivars in different regions according to the breeding objective.

18. The breeding method according to claim 4, wherein in step 3), the process of the selecting further comprises: conducting off-site identification and selection, and selecting a strain with traits better than those of dominant cultivars in different regions according to the breeding objective.

19. The breeding method according to claim 5, wherein in step 3), the process of the selecting further comprises: conducting off-site identification and selection, and selecting a strain with traits better than those of dominant cultivars in different regions according to the breeding objective.

20. The breeding method according to claim 1, wherein in step 1), a process of screening or preparing a breeding material comprises one or more of hybridization, selfing, marker-assisted selection, radiation mutagenesis, genetic engineering, chromosome engineering, and cell engineering.
